(19) United States
(12) Patent Application Publication
(10) Pub. No.: US 2008/0009815 A1
(43) Pub. Date: Jan. 10, 2008

(54) Vacuum Control System For A Breast Pump

(76) Inventors: Richard W. Grabenkort, Franklin, TN (US); Donald V. Matesi, Wauconda, IL (US); Joseph L. Trznadel, Crystal Lake, IL (US); Sam Shadman, Schaumburg, IL (US); Balagru K. Veloo, Gurnee, IL (US)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 11/775,497

(22) Filed: Jul. 10, 2007

Related U.S. Application Data
(60) Provisional application No. 60/806,886, filed on Jul. 10, 2006.

(51) Int. Cl. A61M 1/06

(52) U.S. Cl. 604/346; 417/415; 604/74

(57) ABSTRACT

A closed-loop vacuum control system for a powered breast pump is the subject of the present disclosure. The vacuum control system includes a source of vacuum for applying a vacuum to a milk collection kit. It also includes a device for setting the vacuum level to be produced by the source of vacuum to a preselected vacuum level. The vacuum control system further includes a sensor for sensing the actual vacuum level being produced by the source of vacuum during operation. A controller continuously compares the preselected vacuum level with the actual vacuum level. A proportional valve is also provided for adjusting the actual vacuum level to correspond to the preselected vacuum level. In this manner, the proportional valve can adjust the actual vacuum level in response to a signal from the controller.

### FIELD OF THE INVENTION

The present invention is directed to a breast pump having a source of vacuum for applying a vacuum to a milk collection kit and, more particularly, to a vacuum control system for adjusting the actual vacuum level to correspond to a preselected vacuum level.

### BACKGROUND OF THE INVENTION

It is generally well known that there are various different types of breast pumps available for use by nursing mothers. The purpose of a breast pump is to permit a nursing woman to express breast milk as necessary or convenient into a suitable milk collection device. In this manner, the breast milk that is collected can later be used for feeding breast milk to a baby.

Generally speaking, there are manual breast pumps, electrically-powered breast pumps, and battery-driven breast pumps. The manual breast pumps are typically inexpensive but, due to the nature of such pumps, the applied vacuum and stroke rate are necessarily uneven. Moreover, the fact that they are manually driven means that operating the pump can be a tiring endeavor.

As for electrically-powered breast pumps, they typically include a source of vacuum such as a piston driven by a motor. These breast pumps are sometimes sized to be relatively portable but they also are frequently quite large for institutional applications. In either case, an electrically-powered breast pump typically provides a much more even vacuum and stroke rate.

With regard to battery-driven breast pumps, they also are characterized by providing a relatively even vacuum and stroke rate. It is also the case that battery-driven breast pumps are usually light weight so as to provide a relatively high degree of portability. In some cases, breast pumps have been offered that can be battery driven or operated on standard household electricity.

In contrast to manual breast pumps, the electrically-powered and battery-driven breast pumps offer the advantage to the nursing woman of being able to hold two milk collection devices with their corresponding breast pump shields in place for pumping both breasts at the same time. However, there are times when a nursing mother may want to utilize only a single milk collection device with its corresponding breast pump shield, i.e., she may want to express breast milk from one breast at a time just as she may be used to doing if she has used a manual breast pump.

Regardless of whether a nursing woman uses an electrically-powered or battery-driven breast pump for single pumping, double pumping, or both, it has been a problem with the existing breast pumps on the market to maintain consistent peak vacuum performance no matter whether the pumps are switched to single or double pumping. This is understandable inasmuch as a second milk collection kit adds air volume which, in turn, serves to reduce the peak vacuum performance that can be achieved. For example, one commercially available breast pump is capable of delivering a maximum vacuum of approximately 250 mmHg in the single pumping mode whereas the same commercially available breast pump can only deliver a maximum vacuum of approximately 210 mmHg in the double pumping mode.

In addition to the foregoing, there is a problem with every commercially available breast pump using piston pump technology in that it is believed they are all incapable of maintaining consistent peak vacuum performance over varying barometric pressure, i.e., as the barometric pressure is reduced at higher altitudes the peak vacuum performance is reduced.

The present disclosure is directed to a vacuum control system for a breast pump which is capable of adjusting the actual vacuum level to correspond to a preselected vacuum level regardless of whether the pump is operating in single pumping mode or double pumping mode and regardless of the barometric pressure and/or the altitude where the pump is being used.

### SUMMARY OF THE DISCLOSURE

The present disclosure is directed to a vacuum control system for a breast pump having a source of vacuum, a vacuum level setting device, an actual vacuum level sensor, a controller, and a proportional valve. The source of vacuum is adapted to apply a vacuum to a milk collection kit, and the vacuum level setting device is adapted to set the level of vacuum to be produced by the source of vacuum to a preselected level. The actual vacuum level sensor is adapted to sense the level of vacuum actually being produced by the source of vacuum during operation, and the controller is adapted to continuously compare the preselected and actual vacuum levels.

With regard to the proportional valve, it is adapted to adjust the actual vacuum level to correspond to the preselected vacuum level in response to a signal from the controller.

In an exemplary embodiment, the source of vacuum is a piston driven by a motor for reciprocating movement within a vacuum cylinder. The motor is operatively connected to the piston to produce the reciprocating movement through a speed reducing gearbox, and the speed reducing gearbox is operatively connected to the piston through a rotary-to-linear crank mechanism. With this arrangement, it will be understood that the rotary-to-linear crank mechanism may be operatively connected to the piston through a shaft.

In another respect, the vacuum level setting device may comprise up and down membrane switches operatively connected to the controller. A push-and-release of either of the up and down membrane switches produces a single 2 mmHg vacuum change in the vacuum level setting for the breast pump. In similar fashion, a push-and-hold of either of the up and down membrane switches serves to produce a series of 10 mmHg vacuum changes.

As for other features, the sensor may comprise a differential vacuum transducer for monitoring the actual vacuum level produced. Specifically, the transducer monitors the actual vacuum level present between the vacuum cylinder and the milk collection kit. Further, the differential vacuum transducer provides feedback to the controller on the actual vacuum level.

Other objects, advantages, and features of the present disclosure will become apparent from a consideration of the following specification taken in conjunction with the accompanying drawings.

### BRIEF DESCRIPTION OF THE DRAWINGS

FIG. 1 is a block diagram of a vacuum control system for a breast pump in accordance with the present disclosure;

FIG. 2 is a front elevational view of a breast pump incorporating the vacuum control system illustrated in the block diagram of FIG. 1;

FIG. 3 is a top plan view of a breast pump incorporating the vacuum control system illustrated in the block diagram of FIG. 1;

FIG. 4 is a bottom plan view of a breast pump incorporating the vacuum control system illustrated in the block diagram of FIG. 1;

FIG. 5 is a rear elevational view of a breast pump incorporating the vacuum control system illustrated in the block diagram of FIG. 1;

FIG. 6 is an internal perspective view of certain electrical components of the breast pump of FIG. 2;

FIG. 7 is a perspective view of mechanical components of the vacuum control system of the present disclosure;

FIG. 8 is a top plan view of a rotary-to-linear crank mechanism for driving a piston in accordance with the present disclosure;

FIG. 9 is a perspective view of a vacuum cylinder together with the associated valves for receiving the driven piston of FIG. 8; and

FIG. 10 is a side elevational view of a stepper motor proportional valve for the vacuum control system of the present disclosure.

### DETAILED DESCRIPTION OF THE PREFERRED EMBODIMENT

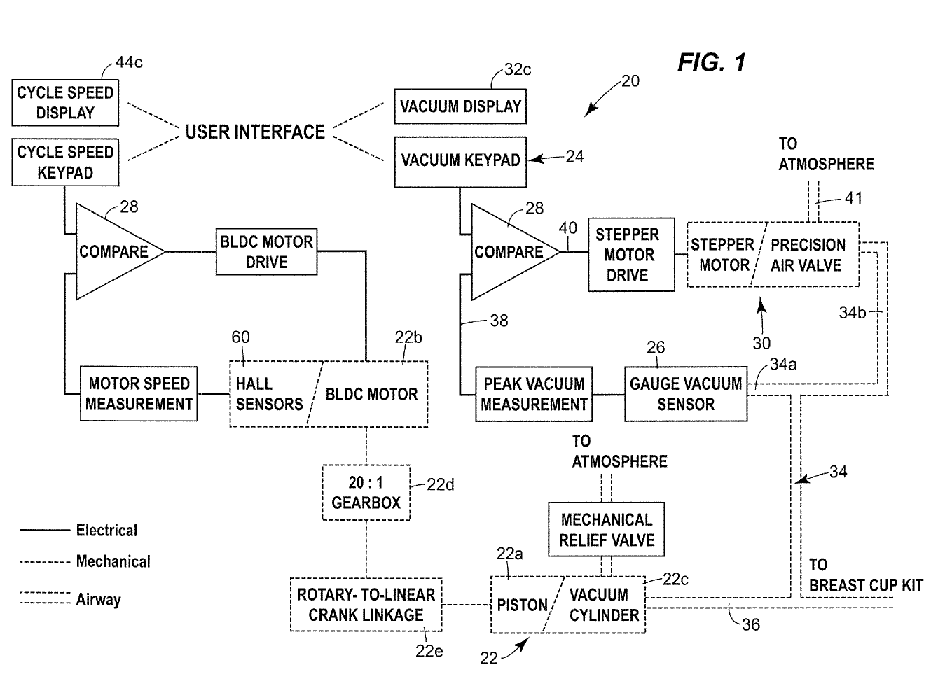

Referring first to FIG. 1, the reference 20 designates generally a vacuum control system for a breast pump in accordance with the present disclosure. The vacuum control system 20 includes a source of vacuum, generally designated 22, for applying a vacuum to a milk collection kit and a device, generally designated 24, for setting the vacuum level to be produced by the source of vacuum 22 to a preselected vacuum level. Also, the vacuum control system 20 includes a sensor 26 for sensing the actual vacuum level produced by the vacuum source 22 during operation.

In addition, the vacuum control system 20 includes a controller 28 for continuously comparing the preselected vacuum level with the actual vacuum level. It still further includes a proportional valve, generally designated 30, for adjusting the actual vacuum level so that it will correspond to the preselected vacuum level in response to an electronic signal from the controller 28. Accordingly, the vacuum control system 20 will be understood to comprise a closed loop system which is capable of providing a precise vacuum during operation.

Still referring to FIG. 1, and the vacuum source 22 includes a piston 22a driven by a motor 22b for reciprocating movement within a vacuum cylinder 22c. The motor 22b is operatively connected to the piston 22a through a suitable speed reducing gear box 22d, and the speed reducing gearbox 22d is, in turn, operatively connected to the piston 22a through a rotary-to-linear crank mechanism 22e. As will be described in greater detail below, the rotary-to-linear crank mechanism 22e is operatively connected to the piston 22a through a shaft.

The motor 22b turns the rotary-to-linear crank mechanism 22e through the speed reducing gearbox 22d in one direction only. The output of the speed reducing gearbox 22d provides the required torque to drive the piston 22a within the vacuum cylinder 22c. The piston 22a is driven within the vacuum cylinder 22c to achieve the desired operating vacuum profile.

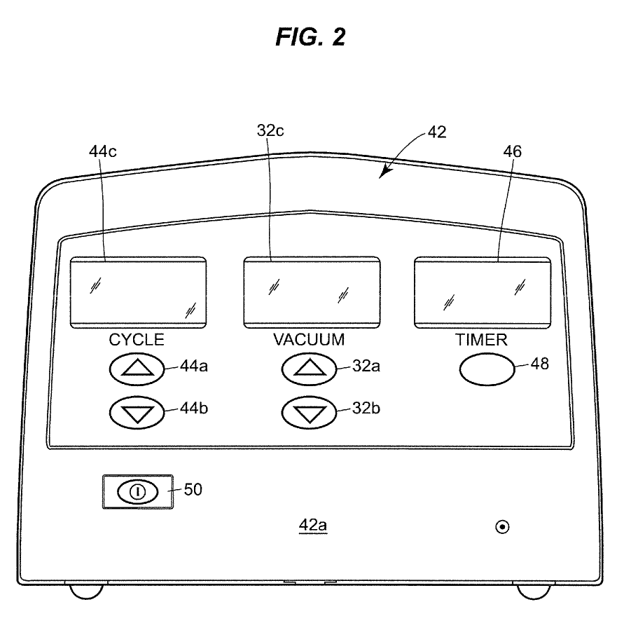

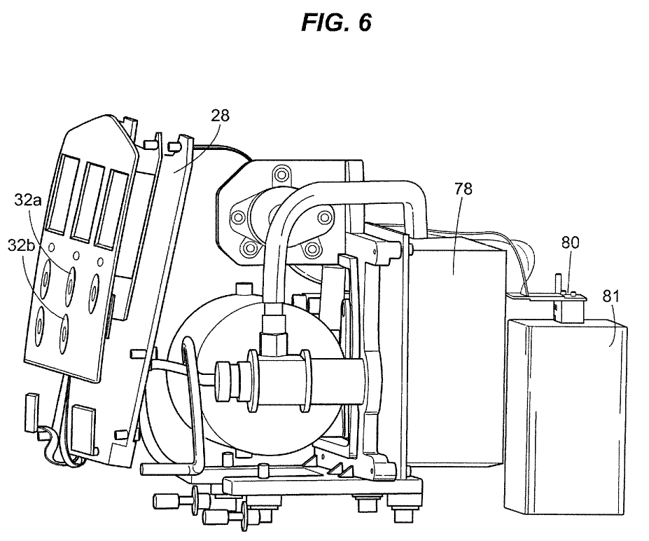

Referring to FIGS. 2 and 6, the vacuum setting device 24 includes up and down membrane switches 32a and 32b operatively connected to the controller 28. The controller 28 is preferably configured such that a push-and-release of either of the up and down membrane switches 32a and 32b produces a single 2 mmHg vacuum change whereas a push-and-hold of either of the up and down membrane switches 32a and 32b produces a series of 10 mmHg vacuum changes. As a result, it is possible to make either fine corrections with the push-and-release functionality or gross corrections with the push-and-hold functionality.

As for the sensor 26, it preferably comprises a differential vacuum transducer for monitoring the actual vacuum level being produced. The differential vacuum transducer 26 monitors the actual vacuum level through an airway or tube 34 in communication with an airway or tube 36 extending from the vacuum cylinder 22c to a milk collection kit, i.e., it monitors the actual vacuum level between the cylinder 22c and the milk collection kit. In addition, the differential vacuum transducer 26 provides feedback to the controller 28 through an electrical line 38.

Referring to FIGS. 1 and 6, the controller 28 comprises a circuit board assembly operatively connected to the sensor 26 and the proportional valve 30. It has been noted that the sensor 26 comprises a differential vacuum transducer which provides feedback through an electrical line 38, and it will be appreciated that the proportional valve 30 is operated through a suitable electrical line 40 (see FIG. 1). In both cases, the electrical lines 38 and 40 send the requisite electrical signals so the preselected and actual vacuum levels can be matched.

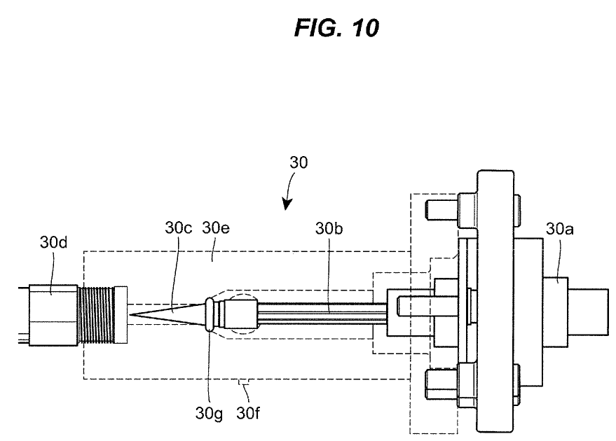

If the comparison shows a difference more than a preprogrammed amount, the proportional valve 30 will cause the actual vacuum level to be adjusted in response to a signal from the controller 28. The proportional valve 30 comprises an air flow valve (see FIG. 10) communicating with the ambient barometric conditions to regulate the amount of ambient air drawn into the system by the vacuum source 22 through, e.g., an airway or tube 41. In this connection, the proportional valve 30 may take the form of a stepper motor 30a having a shaft 30b supporting a cone-shape pin 30c adjacent to a port 30d leading to the vacuum cylinder 22c.

As will be appreciated from FIG. 10, the port 30d leading to the vacuum cylinder 22c is within a valve housing 30c having an opening 30f to ambient barometric conditions wherein the shaft 30b and cone-shaped pin 30c are both within the valve housing 30e. A seal 30g is suitably disposed about the cone-shaped pin 30c adjacent the shaft 30b, and the stepper motor 30a controls the movement of the cone-shaped pin 30c toward and away from the port 30d leading to the vacuum cylinder 22c. In particular, it has been found advantageous for the stepper motor 30a to control the movement of the cone-shaped pin 30c relative to the port 30d by 0.0001 inch increments to provide precise control of the amount of ambient air drawn into the system.

With the foregoing, it will be appreciated that the vacuum control system 20 is a closed loop system capable of maintaining a preselected vacuum level for a breast pump independent of varying ambient barometric conditions.

Referring to FIG. 2, the vacuum control system 20 is well adapted for use in a breast pump of the type illustrated and generally designated 42. The breast pump 42 preferably includes a front panel 42a which includes the up and down membrane switches 32a and 32b for providing the nursing mother an easy manner in which to control the vacuum in either 2 mmHg increments by utilizing the push-and-release functionality or a series of 10 mmHg increments by utilizing the push-and-hold functionality of the pump. Also, there is a vacuum LCD 32c immediately above the membrane switches 32a and 32b to show the vacuum level selected by the nursing mother.

As for other functionality, the breast pump 42 includes another set of up and down membrane switches 44a and 44b for setting the cycle speed. There is also a cycle speed LCD 44c above the membrane switches 44a and 44b. Further, the front panel 42a of the breast pump 42 also includes a session timer LCD 46 and a session timer reset membrane button 48.

Still additionally, the breast pump 42 may advantageously include a membrane button 50 for initiating start up of the pump and placing the pump in a standby mode (“off”) after it has been used.

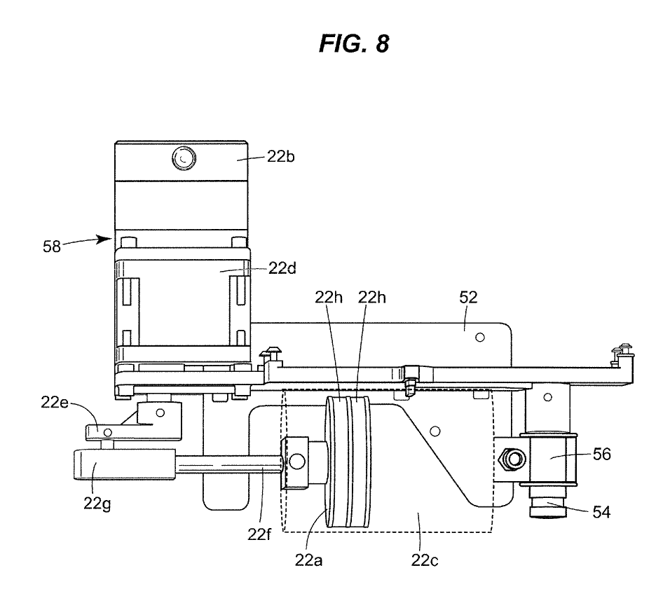

When the membrane button 50 is pushed, the breast pump 42 will automatically start at a minimum preprogrammed vacuum level of 30 mmHg and a maximum preprogrammed pumping cycle speed of 80 cycles/min. Subsequently, the user can adjust the cycle speed and/or adjust the vacuum level using the membrane switches 44a, 44b and 32a/32b, respectively. Preferably, the breast pump 42 is programmed to have a vacuum range of 30 mmHg-250 mmHg developed by the vacuum source 22. The vacuum source 22 may suitably include a 24 V brushless DC (BLDC) motor 22b and a 20:1 speed reducing gearbox 22d. Referring to FIG. 8, the motor 22b drives the piston 22a through a piston shaft 22f and piston shaft link 22g connected, respectively, to the piston 22a and the rotary-to-linear crank mechanism 22e.

Still referring to FIG. 8, it will be seen that the piston 22a disposed within the vacuum cylinder 22c may include one or more piston seals 22h. The piston seals 22h will be understood to be in contact with the inner wall of the vacuum cylinder 22c to cause a vacuum to be drawn as the piston 22a moves from right to left in FIG. 8. The rotary-to-linear crank mechanism 22e and piston shaft link 22g impart linear movement to the piston 22a within the vacuum cylinder 22c.

More specifically, the piston shaft link 22g is coupled to the rotary-to-linear crank mechanism 22e off-center to the rotation of the motor shaft. This serves to impart linear reciprocating motion to the piston shaft 22f and the piston 22a. Further, the vacuum cylinder 22c is coupled to the base of the breast pump 42 through a mounting plate 52 that supports a shaft 54 in generally spaced relation as shown in FIG. 8. The shaft 54 receives a collar 56 to which the vacuum cylinder 22c is connected at the end opposite the piston shaft 22f. With this arrangement, the vacuum cylinder 22c can freely pivot to maintain linear alignment with the piston shaft 22f.

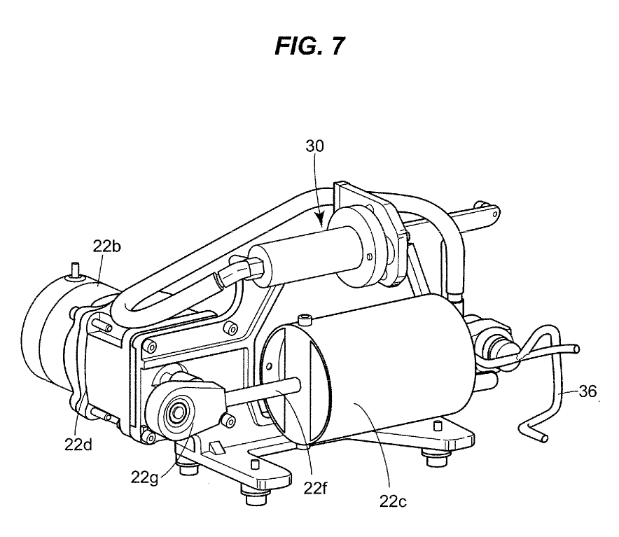

As will be appreciated, the pivoting movement of the vacuum cylinder permitted by the collar 56 mounted on the shaft 54 maintains linear alignment with the piston shaft 22f and the piston 22a for each revolution of the rotary-to-linear crank mechanism 22e. Under hardware/software control, this rotary-to-linear actuator 22e moves the piston 22a with a fixed stroke length and adjustable cycle speed toward the motor/gearbox assembly 58 comprised of the motor 22b and the speed reducing gearbox 22d to increase the vacuum and away from the motor/gearbox assembly 58 to reduce the vacuum. In this connection, the vacuum produced by movement of the piston 22a within the vacuum cylinder 22c will be available to the milk collection kit through a suitable airway or tubing 36 leading from the vacuum cylinder 22c (see FIG. 7).

In the present disclosure, the sensor 26 and proportional valve 30 are disposed within a closed-loop system that maintains consistent pump performance in both single and double pumping modes under varying ambient barometric conditions. The ability to maintain closed-loop control of the vacuum amplitude is implemented as a result of the proportional valve 30 and the differential vacuum transducer 26 and which are in electrical communication with the controller 28. As previously noted, the proportional valve 30 comprises a stepper motor 30a, a valve housing 30e with a defined orifice pathway in the form of the port 30d which cooperates with the cone-shaped pin 30c, and an opening to atmosphere 30f.

With this arrangement, the cone-shaped pin 30c attached to the stepper motor 30a by means of the shaft 30b is used to precisely regulate the amount of ambient air flowing into the vacuum system. Also, the electronic differential vacuum transducer 26 is used to monitor gauge vacuum amplitude relative to ambient barometric pressure and provide feedback to the controller 28. The controller 28 compares the gauge vacuum reading to the desired vacuum and adjusts the proportional valve 30 so that the desired peak vacuum level is achieved.

The vacuum system initial air volume at ambient pressure or zero vacuum is defined by the position of the piston 22a nearest to the output of the vacuum cylinder 22c, the status of the proportional valve 30, the airway or tube 34 from the proportional valve 30 to the vacuum cylinder 22c, the airway or tube 34a from the output of the vacuum cylinder 22c to the vacuum transducer 26 and the airway or tube 36 from the vacuum cylinder output to the pump vacuum port.

In single pumping mode, one milk collection kit is connected to the breast pump 42 and the initial air volume is less than that for double pumping utilizing two milk collection kits. There is automatic compensation for variation in milk collection kit volume as a result of measurement of system vacuum and subsequent regulation of vacuum via of the proportional valve 30 in a closed-loop control manner by the controller 28. In a piston pump, change in vacuum is directly proportional to atmospheric pressure multiplied by the ratio of vacuum system initial volume to final volume so it follows there is at least the potential for varying output peak vacuum for a fixed piston stroke length as atmospheric pressure varies. In the present disclosure, the compensation is achieved over a barometric range of 0.82 atm/83 kPa to 1.05 atm/106 kPa.

Another advantage of the present disclosure and its system vacuum measurement scheme is the ability to alert the nursing mother if there is a leak in the milk collection kits connected to the breast pump 42. A “Check Kit” text message is visible in the vacuum LCD 32c if the measured system output vacuum cannot reach the desired vacuum setting and, thus, conditions such as a milk collection kit being disconnected from the vacuum port, an open milk collection kit tubing adapter, or a missing diaphragm in a milk collection kit will elicit display of this kit error message. As a comfort measure, the user cannot change the cycle speed or vacuum settings at any time while this kit error message is being displayed on the vacuum LCD 32c.

If the “Check Kit” message appears under normal pumping conditions for the breast pump 42, the user can remedy the problem within several seconds to maintain current vacuum settings. However, if the problem is not remedied within several seconds, the pump control system will return the vacuum output to its default setting of 30 mmHg as a comfort measure.

Referring to FIG. 1, closed-loop control of cycle speed is achieved with feedback to the controller 28 from Hall effect sensors 60 which are operatively associated with the BLDC motor 22b. The controller algorithm translates motor/gearbox assembly 58 rotation speed and then compares it to the desired pump cycle speed, adjusting the motor voltage drive as may be necessary.

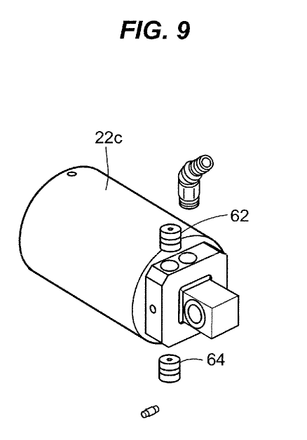

The motor voltage drive software algorithm provides the gradual acceleration/deceleration phases necessary under dynamic loading of the motor/gearbox assembly 58 to achieve an “intermittent” vacuum waveform including a “rest” phase at ambient barometric pressure or zero gauge vacuum, vacuum rise time to peak vacuum amplitude, and vacuum fall time back to ambient barometric pressure for each vacuum cycle. The rest phase of the vacuum cycle at ambient barometric pressure is achieved with a 0.1 psi mechanical one-way check valve 62 mounted on the vacuum cylinder 22c that exhausts the vacuum cylinder to ambient air only after peak vacuum is reached and the piston 22a moves toward the vacuum cylinder output, i.e., toward the end of the vacuum cylinder 22c opposite the piston shaft 22f (compare FIGS. 8 and 9).

For this purpose, a 5.5 psi mechanical relief valve 64 may be mounted on the vacuum cylinder 22c at the vacuum output port in order to prevent vacuum cylinder output from exceeding 300 mmHg.

The vacuum front panel controls, i.e., membrane switches 32a and 32b, are independent of the cycle speed front panel controls, i.e., membrane switches 44a and 44b. Step changes over the peak vacuum range of 30-250 mmHg are made possible using the up and down membrane switches 32a and 32b wherein a single push-and-release results in a target 2 mmHg change and a push-and-hold results in one or more target 10 mmHg changes. The vacuum LCD 32c comprises a graphic liquid crystal display positioned above the vacuum level controls 32a and 32b providing the user with information in two different formats. The graphical liquid crystal display presents a bar graph representing 0-100% of the available 30-250 mmHg peak vacuum range changes in response to use of the vacuum level controls 32a and 32b whereas a numerical display of the percentage facilitates observing the vacuum setting. As for the numerical display, the setting can be used for future pumping sessions and/or communication to a lactation consultant.

The cycle speed front panel controls 44a and 44b are independent of the vacuum level controls 32a and 32b. Step changes in cycles/min over the range of 30-80 cycles/min are made possible using the up and down membrane switches 44a and 44b wherein a single push-and-release results in a target 1 cycle/min change and a push-and-hold results in one or more target 10 cycles/min changes. The cycle speed LCD 44c also comprises a graphic liquid crystal display positioned above the cycle speed controls 44a and 44b providing the user with information in two different formats. The graphical liquid crystal display presents a bar graph representing 0-100% of the available 30-80 cycles/min range changes in response to use of the cycle speed controls 44a and 44b whereas a numerical display of actual cycle speed facilitates observing the cycle speed setting. As for the numerical display, the setting can be used for future pumping sessions and/or communication to a lactation consultant.

As shown in FIG. 2, the session timer LCD 46 may comprise a digital clock format to facilitate a nursing mother following a pumping protocol by indicating the amount of time elapsed during the current pumping session. The nursing mother will turn on the breast pump 42 by pressing the standby membrane button 50 on the front panel of the pump. Immediately thereafter pumping will start at the default settings for the breast pump 42 of 30 mmHg vacuum and 80 cycles/min with the elapsed timer counting up starting from 00:00 (minutes: seconds).

The nursing mother has the option of using the session timer reset membrane button 48 to reset the session timer to 00:00. The elapsed session timer LCD 46 keeps track of the total pumping time; however, an automatic shut-down mode of operation is enabled in the event that the elapsed session timer reaches 60 minutes of continuous run time. This automatic shut-down feature is intended to help guard against excessive pumping and/or unattended operation of the breast pump 42.

After the session timer reaches 60:00, the pump vacuum and cycle speed are reduced to 0 mmHg and 0 cycles/min, respectively, the pump returns to a standby mode and turns off.

The breast pump 42 can be electrically powered over a range of line power voltages (100-240 VAC, 50/60 Hz) and socket configurations encountered throughout the world. It will, of course, be understood that country-specific, detachable power cord sets for the breast pump 42 can be provided as needed. When the breast pump 42 is connected to an electrical outlet, an AC power plug icon is visible in the session timer LCD 46 to indicate the connection to the user.

If desired, the breast pump 42 can be battery-operated with a rechargeable Li-Ion battery pack as an optional accessory. With this optional accessory, the battery capacity and charging status are indicated with icons visible in the session timer LCD 46.

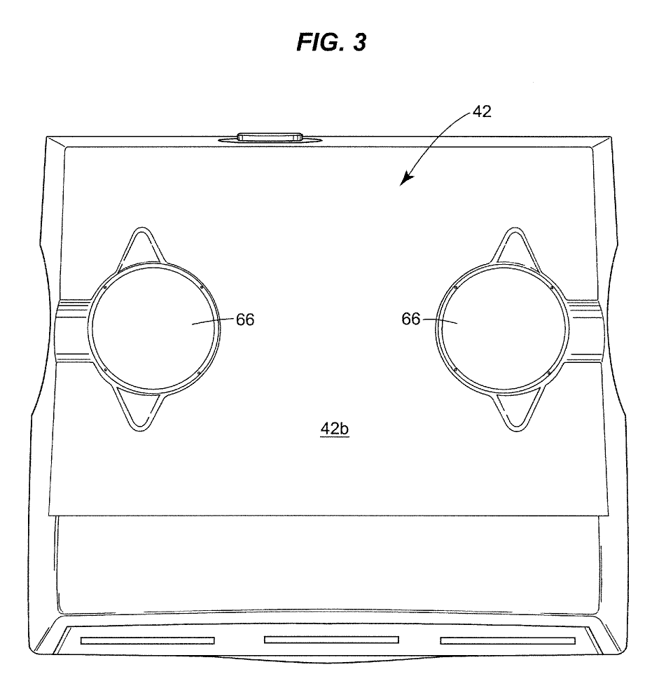

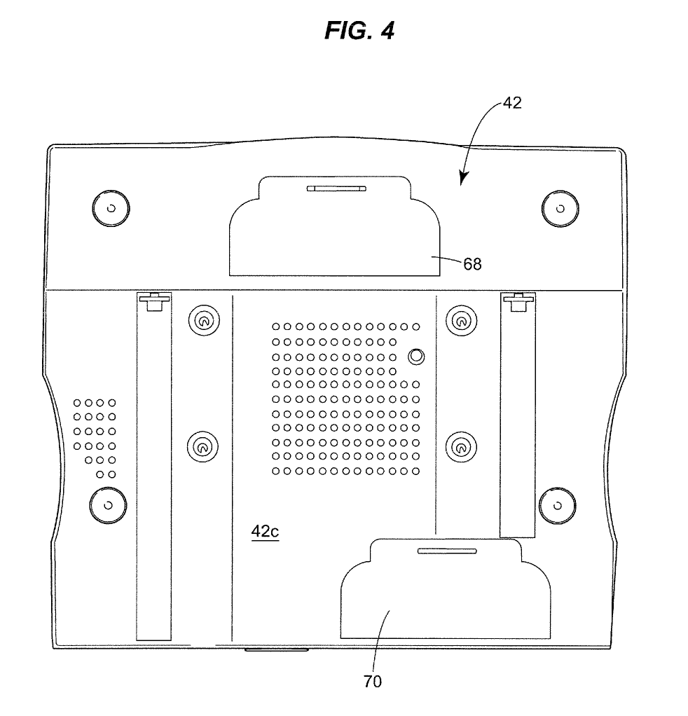

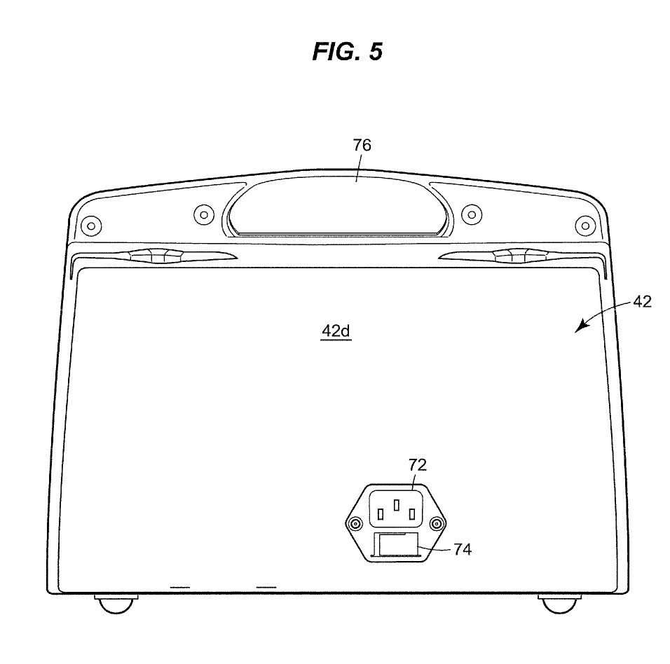

Referring to FIG. 3, the breast pump 42 may include a pair of accessory bottle and breast flange holders 66 on a top surface 42b. It will also be seen that the breast pump 42 may include a diagnostic port door 68 as well as a Li-Ion battery door 70 on a bottom surface 42c where the pump can be battery operated (see FIG. 4). Referring to FIG. 5, the breast pump 42 may also include an AC power appliance inlet 72, a fuse cover 74, and a handle 76 on a rear surface 42d.

As shown in FIG. 6, the breast pump 42 will be seen to include a power supply circuit board assembly 78 and a battery interface circuit board assembly 80 for connection to the rechargeable battery pack 81. FIG. 7 more fully illustrates the closed-loop vacuum mechanism including the motor 22b, the speed reducing gearbox 22d, the piston shaft link 22g, the piston shaft 22f, the vacuum cylinder 22c, the proportional valve 30, and the various airways or tubes. As will be appreciated, FIG. 8 is essentially a top plan view of FIG. 7 with the proportional valve and airways or tubes removed.

While in the foregoing there has been set forth a detailed description of the present disclosure, it will be appreciated by those skilled in the art that the details herein given may be varied without departing from the true spirit and scope of the appended claims.

We claim:

1. A vacuum control system for a breast pump, comprising:
a source of vacuum for applying a vacuum to a milk collection kit;
a device for setting the vacuum level to be produced by the source of vacuum to a preselected vacuum level;
a sensor for sensing the actual vacuum level being produced by the source of vacuum during operation;
a controller for continuously comparing the preselected vacuum level with the actual vacuum level; and
a proportional valve for adjusting the actual vacuum level to correspond to the preselected vacuum level;
the proportional valve adjusting the actual vacuum level in response to a signal from the controller.

2. The vacuum control of claim 1 wherein the source of vacuum is a piston driven by a motor for reciprocating movement within a vacuum cylinder.

3. The vacuum control of claim 2 wherein the motor is operatively connected to the piston through a speed reducing gear box.

4. The vacuum control of claim 3 wherein the speed reducing gear box is operatively connected to the piston through a rotary-to-linear crank mechanism.

5. The vacuum control of claim 4 wherein the crank mechanism is operatively connected to the piston through a shaft.

6. The vacuum control of claim 1 wherein the setting device comprises up and down membrane switches operatively connected to the controller.

7. The vacuum control of claim 6 wherein a push-and-release of either of the up and down membrane switches produces a single 2 mm Hg vacuum change.

8. The vacuum control of claim 6 wherein a push-and-hold of either of the up and down membrane switches produces a series of 10 mm Hg vacuum changes.

9. The vacuum control of claim 2 wherein the sensor comprises a differential vacuum transducer for monitoring the actual vacuum level produced.

10. The vacuum control of claim 9 wherein the transducer monitors the actual vacuum level between the vacuum cylinder and milk collection kit.

11. The vacuum control of claim 10 wherein the transducer provides feedback to the controller on the actual vacuum level.

12. A closed loop vacuum control system for maintaining a preselected vacuum level for a breast pump independent of at least one of varying barometric conditions dependent upon operation of the breast pump at varying altitudes, and varying air volume conditions dependent upon operation of a breast pump in association with one milk collection kit for a single pumping mode versus operation of the breast pump in association with two milk collection kits for a double pumping mode, comprising:
a source of vacuum for applying a vacuum to a milk collection kit;
a device for setting the vacuum level to be produced by the source of vacuum to a preselected vacuum level;
a sensor for sensing the actual vacuum level being produced by the source of vacuum during operation;
a proportional valve for adjusting the actual vacuum level to correspond to the preselected vacuum level; and
a controller for continuously comparing the preselected vacuum level with the actual vacuum level, the controller including a circuit board assembly operatively connected to the sensor and the proportional valve;
the proportional valve adjusting the actual vacuum level in response to a signal from the controller.

13. The closed loop vacuum control of claim 12 wherein the proportional valve comprises an air flow valve communicating with the ambient barometric conditions to regulate the amount of ambient air drawn into the system by the source of vacuum.

14. The closed loop vacuum control of claim 13 wherein the source of vacuum is a piston within a vacuum cylinder and the proportional valve is a stepper motor having a shaft supporting a cone-shaped pin adjacent a port leading to a vacuum cylinder.

15. The closed loop vacuum control of claim 14 wherein the port leading to the vacuum cylinder is within a valve housing having an opening to ambient barometric conditions and the shaft and the cone-shaped pin are both within the valve housing.

16. The closed loop vacuum control of claim 15 including a seal disposed about the cone-shaped pin adjacent the shaft, the stepper motor controlling the movement of the cone-shaped pin toward and away from the port leading to the vacuum cylinder.

17. The closed loop vacuum control of claim 16 wherein the stepper motor controls the movement of the cone-shaped pin relative to the port by 0.0001 inch increments to provide precise control of the amount of ambient air drawn into the system.

18. A closed loop vacuum control system for maintaining a preselected vacuum level for a breast pump independent of at least one of varying barometric conditions dependent upon operation of the breast pump at varying altitudes, and varying air volume conditions dependent upon operation of a breast pump in association with one milk collection kit for a single pumping mode versus operation of the breast pump in association with two milk collection kits for a double pumping mode, comprising:
a source of vacuum for applying a vacuum to a milk collection kit including a piston driven by a motor for reciprocating movement within a vacuum cylinder;
a device for setting the vacuum level to be produced by the motor driven piston to a preselected vacuum level including up and down membrane switches;
a sensor for sensing the actual vacuum level being produced by the motor driven piston during operation including a differential vacuum transducer;
a proportional valve for adjusting the actual vacuum level to correspond to the preselected vacuum level including an adjustable air flow valve; and
a controller for continuously comparing the preselected vacuum level with the actual vacuum level including a circuit board assembly;
the proportional valve adjusting the actual vacuum level in response to a signal from the controller.

19. The closed loop vacuum control system of claim 18 wherein the motor is operatively connected to the piston through a speed reducing gear box, the speed reducing gear box is operatively connected to the piston through a rotary-to-linear crank mechanism, and the crank mechanism is operatively connected to the piston through a shaft.

20. The closed loop vacuum control system of claim 18 wherein the up and down membrane switches are operatively connected to the controller, a push-and-release of the membrane switches produces a single 2 mm Hg vacuum change, and a push-and-hold of the up and down membrane switches produces a series of 10 mm Hg vacuum changes.

21. The closed loop vacuum control system of claim 18 wherein the differential vacuum transducer i) monitors the actual vacuum level produced by the motor driven piston, the actual vacuum level being monitored between the vacuum cylinder and the milk collection kit, and ii) provides feedback on the actual vacuum level to the controller.
